(19) United States
(12) Patent Application Publication
(10) Pub. No.: US 2009/0187161 A1
(43) Pub. Date: Jul. 23, 2009

(54) Sheath and stomach tube

(76) Inventor: Thomas Clyde Hatch, Lexington, KY (US)

Correspondence Address: Thomas Clyde Hatch, 757 Rose Hurst Way, Lexington, KY, 40515, US

(21) Appl. No.: 12/009,419

(22) Filed: Jan. 18, 2008

(51) Int. Cl. A61M 31/00

(52) U.S. Cl. 604/516

(57) ABSTRACT

The embodiments of the present invention are directed to devices and methods for administering oral fluids to animals aseptically. The devices features a sheath having a first surface and a second surface. The sheath is capable of assuming two positions. The sheath is moved from the first position into the second position by inverting it over a stomach tube as the stomach tube is inserted into the mouth and esophagus of an animal. A fluid is then administered through the sheath and stomach tube to the animal.

### CROSS-REFERENCE TO RELATED APPLICATIONS

none

### STATEMENT REGARDING FEDERAL SPONSORSHIP

The present invention was not made or developed with Federal sponsorship.

### BACKGROUND OF THE INVENTION

This invention pertains to an insertion sheath and a separate stomach tube for the delivery of liquid into the stomach of an animal and a method for use.

### DESCRIPTION OF PRIOR ART

A newborn animal, such as a calf, needs to ingest colostrum immediately after birth to obtain the maternal antibodies that will protect it from disease. However, on modern dairy farms, calves are often removed from their dams before nursing, requiring that colostrum be administered to the calf through artificial means, either a nipple and bottle or a stomach tube. The use of a stomach tube to administer the colostrum or other fluids saves the dairy farmer a significant amount of time. While a calf can nurse through an artificial nipple, this may require an inordinate amount of time when the nursing reflex is weak, which is common in newborn animals.

Young animals also suffer from a high rate of diarrhea, or scours. Scours can be caused by administering too much liquid feed to the animal or by infectious disease pathogens such as E. coli or rotavirus. In either case, the dehydration that occurs as a sequela to scours must be reversed to return the young animal to health. In many instances, fluid therapy with electrolytes must be continued for several days to prevent mortality.

Occasionally, young animals experience milk bloat. This can occur when milk enters the rumen rather than the abomasum and ferments, forming copious quantities of gas. When the gas cannot escape, the pressure can build up and cause death. A properly placed stomach tube can vent such gases and keep an animal from dying.

Though placing a stomach tube in an animal can serve several important purposes, there are obvious drawbacks to the procedure. These drawbacks apply to the insertion of any device into a body cavity. For example, devices of the prior art can easily spread disease from one animal to another if not properly sanitized between uses. It is imperative that multi-use prior art devices be disinfected between uses to prevent horizontal transmission of diseases across animals. No prior art stomach tubes have made use of an invertible sheath to prevent contact between the stomach tube and the inside walls of the body cavity, thereby isolating a potentially-contaminated stomach tube from the mucosal surfaces of the mouth and esophagus. No prior art devices have been designed to minimize friction between the stomach tube and the mucosal lining of the body cavity during the insertion and removal steps. Most instructions on how to use stomach tubes state that the tube should be dipped in a fluid to lubricate the outer surface to make it easier to insert. This is an especially important issue in the case of a dehydrated neonate with scours whose mucosal surfaces may already be semi-dry. A stomach tube must be inserted gently.

This need to insert a device or object gently is also a concern when other body cavities are involved. This is especially the case with the insertion of tampons into the vaginal cavity. U.S. Pat. No. 4,413,986 describes an assembly consisting of a sheath, an insertion tube, a guide tube and an object to be delivered. All components are integrally connected. There is no description of an insertion sheath that is separate from the insertion tube and guide tube. There is no description of a guide tube that is maintained a short distance from the cavity opening to prevent entry of any contaminants into the body cavity as the insertion tube is used to turn the insertion sheath inside out. The '986 patent does not anticipate the use of an invertible sheath with an insertion tube.

U.S. Pat. No. 4,923,440 describes a sterile receptacle for the storage and insertion of an object, such as a tampon or a suppository, into a body cavity. The device is comprised of an insertion sheath, an inner container and an outer container that are integrally attached. Consequently, the inner container or insertion tube, and the outer container or guide tube, can be used only once. The patent does not describe an insertion sheath that is separate from the insertion tube or guide tube. It does not describe an insertion sheath of sufficient length to also function as a tether to facilitate the sheath being turned outside in as it is withdrawn from the body cavity following use, thereby preventing friction or irritation to the mucosal surface of the body cavity during both insertion and removal. This patent does not describe the use of an insertion tube that reduces the friction between its outside surface and the insertion sheath. This patent does not anticipate the use of an invertible sheath for use with a stomach tube.

U.S. Pat. No. 3,681,504 describes the use of guanidine to stimulate closure of the esophageal groove in young suckling ruminants. Receptors in the oral cavity, pharynx and esophagus trigger the closure. The esophageal groove is a continuation of the esophagus as a groove or tube which bypasses the reticulo-rumen area. In young calves and other suckling ruminants, a closed tube is formed when the lips of the groove shorten and invert. This conveys milk or other fluids from the cardia to the omasal canal. The milk runs through the omasum to the abomasum as the reticulo-omasal orifice and the omasal canal simultaneously relax. The rumen is completely by-passed.

Delivery of fluids directly to the omasal-abomasal compartments can be important in neonates. Immunoglobulins from colostrum can be absorbed from the intestine for just twenty-four hours following birth. The sooner the absorption process begins, the more total protective immunoglobulins can be absorbed by the animal. Colostrum delivered directly through a closed esophageal groove to the omasal-abomasal compartments will not encounter a reduced window of time during which absorption occurs in the gastrointestinal tract downstream from the reticulo-rumen area. The more timely delivery of other fluids, such as oral drugs and electrolytes, to the omasal-abomasal compartments can lead to higher circulating drug and electrolyte concentrations in a shorter time span.

In somewhat older animals, bypassing the rumen through means of closing the esophageal groove could result is a lower incidence of milk bloat when milk is fed with a stomach tube.

U.S. Pat. No. 6,213,960 describes the use of an electric current applied through electrodes attached to an endotracheal tube to stimulate closure of the glottic opening.

Chemicals, in addition to guanidine, that can cause closure of the esophageal groove, especially in adult animals include sodium chloride, copper sulfate and sodium bicarbonate. By-passing the rumen can be imperative in maintaining the bioactivity of substances such as vitamins, anti-bloat compounds and amino acids.

### SUMMARY OF THE INVENTION

This invention is based on the novel and surprising concept of using an insertion sheath with a separate multi-use stomach tube for the administration of fluids to or for the removal of gases from an animal. Each insertion sheath has a first surface and a second surface and two positions. In the first position the first surface of the insertion sheath faces inward and the second surface faces outward. In the second position, the first surface faces outward and the second surface faces inward for the portion of the insertion sheath that has been inverted by the stomach tube. The insertion sheath is initially configured in the first position inside the stomach tube. It is moved into the second position by means of moving the stomach tube into the animal in a manner that inverts the sheath, one end of which remains stationary a small distance from the animals mouth. The portion of the sheath that is not inverted can be integrally or temporarily attached to a reservoir containing fluid or can be opened to vent gas when the stomach tube is used on an animal with bloat. After fluids have been administered through the sheath and stomach tube, the sheath can be moved from the second position back to the first position by treating the portion of the sheath that did not turn inside out as a tether. It is pulled out and away from the stomach tube, turning the inverted sheath outside in. A sheath that is returned to the first position can be hygienically disposed of.

A significant amount of friction can occur when an insertion sheath is inverted along the length of a stomach tube inserted through the mouth and esophagus of an animal. The cavity walls of the animal place inward pressure on the inverted sheath that in turn presses against the outside surface of the stomach tube. The friction can be reduced by employing low friction materials such as ptfe in the stomach tube and/or the disposable sheath. A stomach tube is provided that reduces the amount of friction between itself and the insertion sheath by providing three or more raised ridges that run parallel along the length of the stomach tube. These ridges reduce the total area of contact between surface two of the inverted insertion sheath and the outside surface of the stomach tube.

In one embodiment the stomach tube is telescoped inside of a guide tube. The guide tube is anchored by means of a ring muzzle that keeps it a small distance from the mouth of the animal into which the stomach tube is being inserted. The insertion sheath is drawn through and inverted out and back over the end of the stomach tube where it is attached to the guide tube. The user aligns the sheath-covered stomach tube to slide smoothly along the tongue into the throat and down the esophagus of an animal. As the stomach tube is moved forward through the stationary guide tube, the portion of the stomach tube covered by the insertion sheath enters the mouth, esophagus and stomach, moving the insertion sheath from the first position to the second position in the process. As the stomach tube is inserted into the stomach, the near end of the insertion sheath that is attached to the guide tube remains in a fixed position away from the mouth of the animal. The insertion sheath prevents the stomach tube from ever contacting any internal surface of the animal, thereby preventing the spread of disease by repeated use of the multi-use stomach tube. The stomach tube does not have to be disinfected between uses. The insertion sheath isolates the stomach tube completely from the interior cavity of the animal.

The insertion sheath is made with flexible walls. It must be folded to allow for easy passage through the stomach tube. The stomach tube is typically cylindrical in design, with enough interior space to allow the insertion sheath to slide freely within and to allow fluid to flow through it as fluid is administered to the animal.

In instances where the closure of the esophageal groove is desired, chemicals that are known to cause such closure can be stored in a compartment of the insertion sheath that will contact the oral tissues, pharynx tissues and esophageal tissues as the sheath is moved from position one to position two as it is being turned inside out. Closure of the groove can be achieved in a minute or two, after which the fluid to be administered can be allowed to start flowing through the sheath and stomach tube. Only enough chemical to coat the portion of the insertion tube that is inverted and that contacts the mucosal tissues of the animal need be used.

### OBJECTS AND ADVANTAGES

Users continually search for insertion means and devices which overcome deficiencies of prior art devices, which can save time for the producer and are relatively simple to use. It is an object of the present invention to provide a disposable insertion sheath that is separate from the stomach tube and a method for the placement of the stomach tube with the inverted sheath into the esophagus and stomach of an animal to administer fluids or vent gases.

It is a further object of the invention to provide packaging and a delivery method that obviates the need to disinfect the multi-use stomach tube prior to use across more than one animal.

It is a further object of the invention to provide a means to insert the stomach tube without requiring the use of lubrication to prevent friction between the side walls of the body cavity and the device during both insertion into and removal from the body cavity. Instructions for the use of many stomach tubes state that the tubes should be dipped or coated in milk or other fluid to make them slide easier during the insertion process.

It is a further object to provide a compartment in the sheath in which the first surface is coated with a chemical(s) that can cause the esophageal groove to close when contacting receptors in the oral cavity, the pharynx and the tissues of the esophagus.

It is a further object of the invention to provide an insertion sheath that can be returned to its original configuration following administration of the fluid, suitable for easy hygienic disposal

It is a further object of the invention to provide a stomach tube that reduces the friction between its outer surface and the second surface of the insertion sheath while the sheath and stomach tube are being inserted and removed from a body cavity.

It is a further object of the invention to provide for an insertion sheath that can be either integrally attached to a fluid-containing reservoir or can be temporarily connected to such reservoir or remain an open sheath.

Other objectives of the present invention will be apparent from the following detailed description of the preferred embodiments.

### BRIEF DESCRIPTION OF THE DRAWINGS

FIG. 1 is a perspective view of the insertion sheath attached to a fluid reservoir.

FIG. 2 is a cross section end view of the unfolded insertion sheath.

FIG. 3 is a cross section end view of the insertion sheath folded so it can be inserted into the stomach tube.

FIG. 4 is a cross-sectional end view of a stomach tube with raised ridges.

FIG. 5 is perspective view of a semi-flexible stomach tube placed inside a guide tube attached to a muzzle ring which is placed over the muzzle of an animal.

FIG. 6 is a perspective view of a fluid reservoir attached to an insertion sheath which has been placed inside the stomach tube, ready to be inverted or moved from position one to position two.

FIG. 7 is a cross-section side view of the insertion sheath turned inside out by the stomach tube that has been inserted down through the esophagus of a calf to its stomach.

FIG. 8 is a perspective view of an insertion sheath comprising a storage compartment containing a chemical that stimulates closure of the esophageal groove, an additional length through which the fluid to be administered will flow, and the fluid reservoir.

### DESCRIPTION OF THE PREFERRED EMBODIMENTS

The need in the art for an improved fluid delivery system is satisfied in the present invention. The invention provides a means for safely administering fluids orally to animals while reducing or eliminating the possibility of spreading pathogenic organisms.

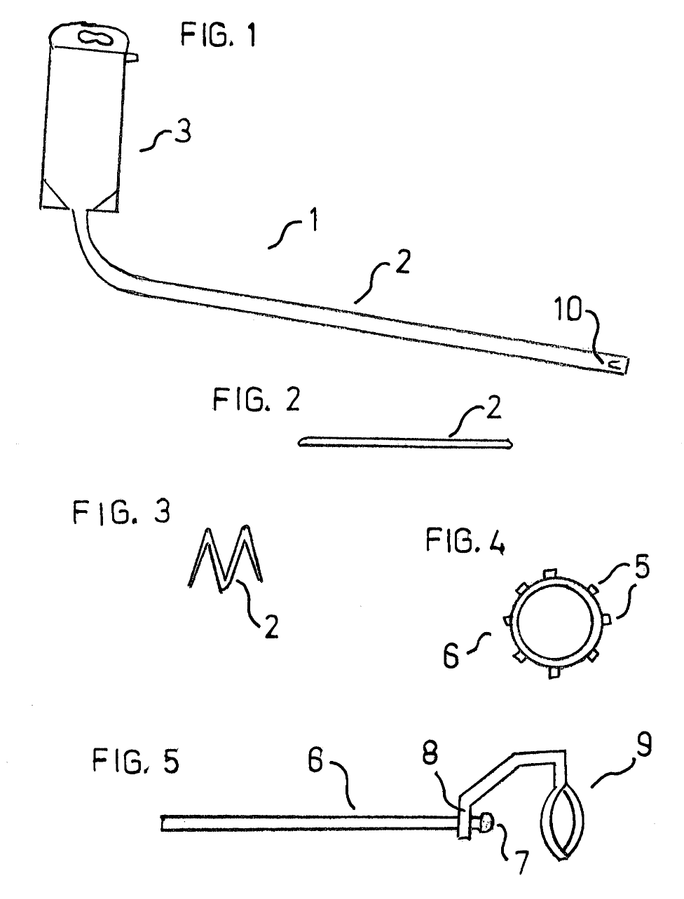

The nature of the invention can be more fully appreciated by reference to the appended drawings, in which a preferred embodiment is depicted. Insertion sheath assembly 1 is shown in FIG. 1. Insertion sheath assembly I is comprised of insertion sheath 2, tape tab 10 at one end of insertion sheath 2 and fluid storage reservoir 3 attached to the opposite end. Insertion sheath 2 can alternatively comprise only the sheath portion that can be attached to a separate fluid reservoir at the time of use or that can be used to vent gasses.

The length of insertion sheath 2 is typically two and one half times the distance from the animal's mouth to the animal's stomach. Tape tabs 10 are used to attach insertion sheath 2 to guide tube 8 of FIG. 5.

The unfolded insertion sheath 2 of FIG. 2 is essentially a lay flat tube, which when opened into a cylindrical shape, is slightly greater in circumference than the stomach tube 6 of FIG. 5. However, in the flat configuration shown in FIG. 2, the insertion sheath 2 will not easily pass through the interior of stomach tube 6 of FIG. 5. Insertion sheath 2 has an inward-facing first surface on the interior and an outward-facing second surface on the exterior when in position one. In position two, the first surface of insertion sheath 2 faces out and the second surface faces in as the sheath is turned inside out.

While insertion sheath 2 can be curled inside of stomach tube 6 of FIG. 5, a preferred embodiment is to have it folded as shown in FIG. 3. The folding pattern shown in FIG. 3 allows the insertion sheath 2 to be easily inserted through stomach tube 6 as it is turned inside out moving from position one to position two and passes more easily over everting ball 7 attached to the end of stomach tube 6. The total circumference of insertion sheath 2 when fully opened is slightly larger than the outside circumference of everting ball 7, which is slightly larger than the circumference of stomach tube 6. The non-everted portion of insertion sheath 2 functions as a tether and is pulled away from the stomach tube 6 following administration of fluid to the stomach to return the insertion sheath 2 from position two to position one.

FIG. 4 depicts a cross section end view of the preferred embodiment of stomach tube 6. The walls of the mouth and esophagus can exert a significant amount of pressure against the first surface of insertion sheath 2, forcing it tightly against the exterior surface of stomach tube 6. This can cause a significant amount of friction that makes it necessary to apply even more force to insert stomach tube 6 through the esophagus toward the stomach. This may make it difficult to differentiate between the resistance caused by the excess friction and any resistance caused by misalignment of stomach tube 6 in the esophagus. The elevated ridges 5 on stomach tube 6 help to minimize such friction. Ridges 5 run the length of stomach tube 6.

FIG. 5 is a perspective view of stomach tube 6, guide tube 8 and muzzle ring 9. Stomach tube 6 has a hollow everting ball 7 attached to the end that is inserted into the mouth and esophagus of the animal. Everting ball 7 serves two purposes. First, everting ball 7 provides a smooth rounded surface over which insertion sheath 2 is turned inside out. Secondly, it is of sufficient diameter to prevent entry of stomach tube 2 into the trachea of the animal. This ensures that stomach tube 2 is properly placed in the esophagus where fluid can be administered to the stomach rather than into the trachea where the fluid would enter the lungs and drown the animal. Muzzle ring 9 is placed over the muzzle of the animal.

FIG. 6 is a perspective view of insertion sheath 2 placed inside stomach tube 6, with tape tabs 10 pulled back over everting ball 7. Tape tabs 10 are attached to stationary guide tube 8. With insertion sheath 2 in this position, muzzle ring 9 can be placed over the muzzle of the animal.

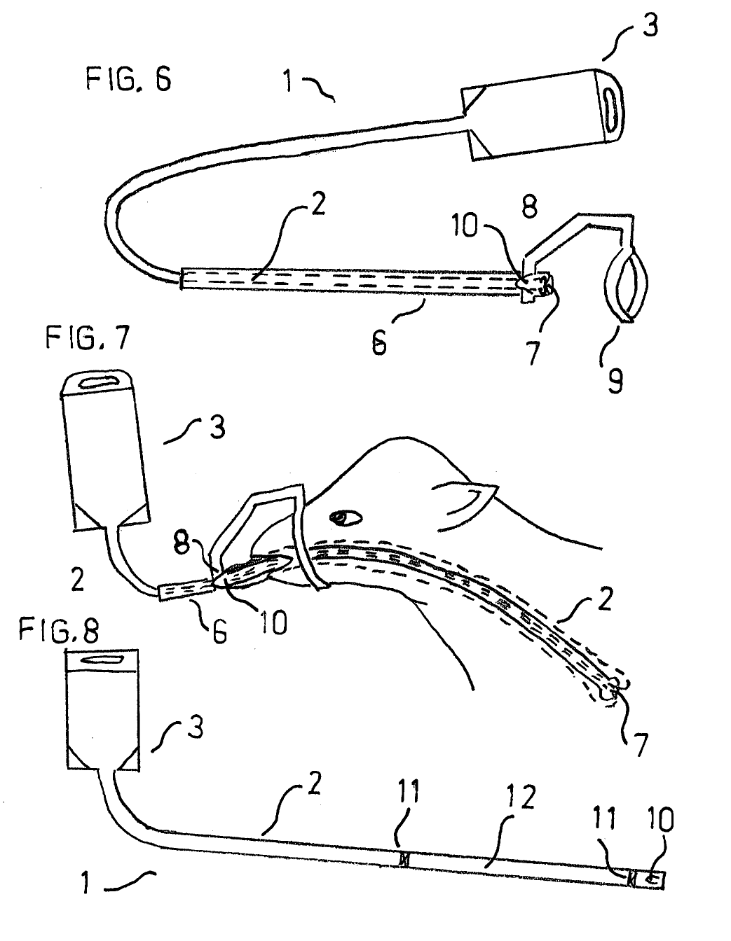

FIG. 7 is a cross section side view of insertion sheath 2 in the second position, turned completely inside out for the portion adjacent to the outside surface of stomach tube 6. As stomach tube 6 is inserted through guide tube 8 into the mouth and through the esophagus of an animal, the tape tabs 10 hold one end of insertion sheath 2 stationary. Insertion sheath 2 is turned inside out or alternatively, moved from position one to position two as stomach tube 6 is inserted to the stomach. When everting ball 7 has passed through the esophagus, fluid reservoir 3 can be raised above the head of the animal and fluid administered through the portion of insertion sheath 2 that remains inside stomach tube 6 and that was never turned inside out. Flow of fluid will cause the folded insertion sheath 2 to partially unfold, thereby enabling such fluid flow. If the storage reservoir 3 can be closed with an airtight seal, it can be squeezed to force fluid out and down through the insertion sheath into the stomach. Otherwise fluid can flow by the pull of gravity as the storage reservoir is held above the head of the animal. Fluid from the storage reservoir 3 can be prevented from prematurely flowing through the insertion sheath 2 by mechanical or other means. Storage reservoir 3 can initially contain either a powder or a fluid. If a powder, water can be added to hydrate it before administration.

FIG. 8 is a perspective view of insertion sheath assembly 1 in an alternate configuration comprising fluid storage reservoir 3, insertion sheath 2, and storage compartment 12. Storage compartment 12 is sealed at each end with breakable seals 11. Seals 11 break as insertion sheath 2 is inverted. Storage compartment 12 contains a chemical(s) that will stimulate closure of the esophageal groove when insertion sheath 2 is inverted and surface one of storage compartment 12 contacts the signaling receptors in the oral cavity, the pharynx and the esophagus. Chemicals that have been shown to cause closure of the esophageal groove include guanidine, sodium chloride, copper sulfate and sodium bicarbonate.

Following administration of fluid from storage reservoir 3 to the animal's stomach, the insertion process is reversed. The portion of insertion sheath 2 that is not positioned inside stomach tube 6 serves as a tether. As it is pulled out and away from the open end of stomach tube 6, stomach tube 6 is likewise drawn from the body cavity and insertion sheath 2 is turned outside in and returned to its first position. After being returned to position one, insertion sheath 2 can be safely disposed of without risk of spreading pathogens from a sick animal. Surface one that contacted the mucosal surfaces of the animal is facing inward on the interior of insertion sheath 2. Withdrawal of insertion sheath 2 as described above will eliminate any irritation to the mucosal surface of the esophagus or mouth as well.

The small distance between guide tube 8 and the mouth of animal ensures that insertion sheath 2 that enters the mouth is not contaminated either by the stomach tube 6 or by the dirty hands of the user.

The length of the insertion sheath 2 and stomach tube 6 is primarily determined by the desired delivery depth. Stomach tube 6 should be one foot longer than the distance from the mouth to the stomach of an animal. Insertion sheath 2 should be approximately 2½ to 3 times the length of stomach tube 6.

Stomach tube 6 is preferably manufactured from semi-flexible plastic hosing that can bend as it is inserted into the mouth and esophagus of an animal but that is sufficiently stiff to turn insertion sheath 2 inside out. Guide tube 8 can be manufactured from a variety of materials, including metal, or plastic, such as nylon, or any combination of these materials to ensure that it is rigid enough to support the stomach tube as it is inserted into the animal. The insertion sheath 2 is preferably manufactured from an elastomeric or flexible plastic sheet, foil or polyfoil that can provide the needed barrier between stomach tube 6 and the mucosal surface of the animal but that can easily be turned inside out. Insertion sheath 2 can be sterilized at the time of manufacture if needed.

The preferred embodiment of the present invention obviates the need to wash stomach tube 6 between uses. It never comes in contact with the animal. It is always isolated from the inside of the mouth and esophagus by the insertion sheath 2 in position two. With prior art devices which most typically included only a stomach tube, it was very difficult to maintain proper sanitation. Even if entire stomach tubes were boiled in water and sterilized, these prior art stomach tubes must still be handled by the user, thereby contaminating them along their entire length as they are manipulated by the user during the insertion process. A typical dirty barnyard environment makes it near impossible to place a traditional stomach tube into the oral cavity and esophagus of an animal without introducing microbes.

A surprising benefit of this invention is the difficulty with which a user would have to make an error that could even lead to spreading of disease between animals. Any contamination of the everted insertion sheath 2 as it is attached to the guide tube 8 does not provide a threat of contamination to the animal because an additional length of the insertion sheath 2 will be turned inside out before entering the mouth.

The use of a separate stomach tube 6 and insertion sheath 2 leads to significant cost savings, both from the material saved by providing a single re-usable stomach tube 6 with multiple insertion sheaths 2 and from the reduced transportation volume associated with having to ship only replacement insertion sheaths 2.

The amount of chemical stored in compartment 12 is just sufficient to coat the inside surface or surface one of compartment 12.

The preferred embodiment of the invention comprises a guide tube 8 attached to a muzzle ring 9. However, the muzzle ring can be dispensed with if the guide tube 8 or the tape tabs 10 of the insertion sheath 2 are grasped and held away from the animal's mouth with one hand while the stomach tube is plunged forward with the other hand, turning the insertion sheath 2 inside out. The most surprising and beneficial element of this invention is the use of a separate stomach tube 6 to turn the insertion sheath 2 inside out.

While a particular embodiment has been described above in connection with emplacement in a calf, it is to be understood that the insertion sheath 2 and stomach tube 6 are suitable for use in connection with all mammals, including but not limited to those of the bovine, ovine, caprine, equine, canine and porcine families, as well as humans.

It will be apparent to those skilled in the art that many modifications and variations may be introduced without departing from the inventive scope of the present teachings.

I claim:

1. A device for administering a fluid to an animal comprising a sheath having a first surface and a second surface, said sheath capable of assuming two positions, in said first position said first surface faces inward defining a first opening and a first chamber, and said second surface faces outward defining a second opening and a second chamber.

2. The device of claim 1 wherein said sheath is used in said second position to administer a fluid to an animal, including a human, and upon completion of the administration of the fluid, said sheath is capable of being returned to said first position and removed from the oral cavity of the animal.

3. The device of claim 1 further comprising a portion of the sheath functioning as a tether element projecting from said second surface to facilitate moving said sheath from said second position to said first position.

4. The device of claim 1 further comprising said sheath attached to a storage reservoir filled with material selected from the group consisting of colostrum, milk, electrolyte solution, oral drugs, vitamins, anti-bloat compounds, amino acids, mineral oil, or any fluid that brings about a physiological change to the animal, or any powder or granule that can be re-hydrated into any of the aforementioned fluids.

5. The device of claim 1 wherein said second chamber receives a stomach tube.

6. The device of claim 3 wherein said sheath cooperates with a stomach tube having an exterior surface and an interior surface, said sheath assuming said second position with said stomach tube occupying said second chamber and said second surface abutting said exterior surface.

7. The device of claim 6 wherein said stomach tube has at least one interior surface defining a passage, said sheath received in said passage in said first position and assuming said second position as said second surface is drawn over in abutting relationship with said exterior surface of said stomach tube.

8. The device of claim 5 further comprising a stomach tube.

9. The device of claim 8 wherein said stomach tube has an interior surface and an exterior surface, said interior surface defining a passage for receiving said sheath in a first position said exterior surface for receiving said sheath in said second position.

10. The device of claim 8 wherein said stomach tube is inserted through a guide tube into the oral cavity of an animal, with said guide tube being maintained apart from the oral cavity of the animal.

11. The device of claim 8 wherein said stomach tube has three or more raised ridges running lengthwise along its exterior surface.

12. The device of claim 3 wherein said sheath functions as a tether element projecting from said second surface, said tether element occupying said passage when a portion of said sheath is in said second position and capable of drawing the inverted portion of said sheath into said first position in cooperation with said implement head as said tether element is withdrawn from said passage.

13. The device of claim 3 wherein said sheath has a temporary storage compartment with breakable seals containing small volumes of a fluid selected from the group consisting of guanidine, sodium chloride, copper sulfate and sodium bicarbonate.

14. The device of claim 1 wherein said sheath is comprised of one or more materials selected from the group consisting of metal foils, polyfoils or plastic.

15. The device of claim 8 wherein said stomach tube is comprised of one or more materials selected from the group consisting of metals, plastics or rubber.

16. A method of administering a fluid to an animal comprising the steps of
a.) providing a device having a sheath, said sheath having a first surface and a second surface, said sheath capable of assuming two positions, in said first position said first surface faces inward toward itself defining a first opening and a first chamber and said second surface faces outward; and
b.) opening said one end of sheath and manipulating said sheath into said second position; and
c.) using said sheath in second position to administer a fluid to an animal and to provide a barrier between the stomach tube and the mucosal surfaces of the animal; and
d.) manipulating said sheath back into said first position for disposal.

17. The method of claim 16 wherein said sheath cooperates with a stomach tube having an exterior surface and an interior surface, said sheath assuming said second position with said stomach tube occupying said second chamber and said second surface abutting said exterior surface and said sheath is drawn over said stomach tube as said sheath assumes said second position.

18. The method of claim 17 wherein said stomach tube has at least one interior surface defining a passage, said sheath received in said passage in said first position and assuming said second position as said second surface is drawn over in abutting relationship with said exterior surface of said stomach tube.

19. The method of claim 18 wherein the portion of said sheath that does not assume position two functions as a tether element capable of drawing the portion of said sheath in position two back into the first position in cooperation with said stomach tube as said tether element is withdrawn from said passage.

20. The method of claim 17 wherein said stomach tube has an interior surface and an exterior surface, said interior surface defining a passage for receiving said sheath in a first position said exterior surface for receiving said sheath in said second position, said method further comprising the step of storing at least one sheath in said passage.
